(19) United States
(12) Patent Application Publication
(10) Pub. No.: US 2016/0213632 A1
(43) Pub. Date: Jul. 28, 2016

(54) Memory Performance and Prevention and Treatment of Neurodegenerative Diseases

(71) Applicant: Max Planck Florida Institute for Neuroscience, Jupiter, FL (US)

(72) Inventors: Ryohei Yasuda, Jupiter, FL (US); Erzsebet Szatmari, Jupiter, FL (US)

(21) Appl. No.: 15/006,753

(22) Filed: Jan. 26, 2016

Related U.S. Application Data
(60) Provisional application No. 62/107,803, filed on Jan. 26, 2015.

(51) Int. Cl. A61K 31/197

(52) U.S. Cl. CPC A61K 31/197

(57) ABSTRACT

The present technology relates to improvement of memory performance and the prevention or treatment of brain disorders associated with memory loss. In particular, the invention relates to the use of Centaurin-α1 (CentA1), CentA1 activating proteins, and CentA1 effector proteins as therapeutic targets to improve memory and cognitive performance in a normal subject and/or in a subject with neurological disorders associated with memory and/or cognitive impairments, such as Alzheimer's disease.

### CROSS REFERENCE TO RELATED APPLICATIONS

The present application claims priority to U.S. Provisional Patent Application No. 62/107,803, filed on Jan. 26, 2015, which is incorporated by reference herein in its entirety.

### FIELD

The present technology relates to improvement of cognitive and memory performance and the prevention or treatment of brain disorders associated with memory loss. In particular, the invention relates to the use of centaurin-α1 (CentA1), CentA1 activating proteins, and CentA1 effector proteins as therapeutic targets to improve memory and cognitive performance in a normal subject and/or in a subject with neurological disorders associated with memory and/or cognitive impairments, such as Alzheimer's disease.

### BACKGROUND

Neurodegeneration refers to the progressive loss of structure or function of neurons and includes the death of neurons. Neurodegeneration can occur as a part of the aging process and can be caused by neurodegenerative diseases including amyotrophic lateral sclerosis (ALS), Parkinson's disease, Alzheimer's disease and Huntington's disease.

Alzheimer's disease (AD) is the most common form of neurodegeneration leading to dementia. According to ADI World Alzheimer Report 2012 the global prevalence of AD is expected to increase to 135 million people by 2050. AD is a multifactorial disorder characterized by severe cognitive decline, memory loss, language deterioration, executive and visuospatial dysfunction, and personality and mood changes, including apathy, agitation, anxiety and depression (Mucke, 2009). Despite 100 years of research, the cause and progression of AD are only partially understood and there is no treatment available. There are four main hypotheses regarding the etiology of AD: the cholinergic, the amyloid, the tau and the vascular hypothesis. According to the amyloid hypothesis of AD (Hardy and Selkoe, 2002), β-amyloid peptide (Aβ), derived from β-amyloid precursor protein (APP), is the major factor in Alzheimer's disease (AD) pathogenesis (Goldsworthy and Vallence, 2013; Jin et al., 2011; Li et al., 2011; Sheng et al., 2012; Teich and Arancio, 2012; Yu and Lu, 2012; Yu et al., 2012; Zahs and Ashe, 2013). Aβ can be polymerized to many different forms from soluble monomer and oligomer to larger fibril-like structures (Selkoe, 2008; Yang et al., 2013). It has been reported that the soluble oligomeric form of Aβ is pathogenic and causes the AD-like cellular phenotypes including dendritic spine loss, impairment in synaptic plasticity and spine structural plasticity in dissociated cultures and slices (Mucke and Selkoe, 2012). These neuronal dysfunctions are thought to be signs of early AD. A number of studies have identified signaling pathways initiated by Aβ. It has been reported that PrP (Lauren et al., 2009) and LilrB2 (Kim et al., 2013) are potential receptors of Aβ that mediate AD pathology. NMDA receptors also appear to play a role in Aβ-induced neuronal dysfunction. Several intracellular signaling molecules, including GSK3β (Ma, 2013), Ras (Megill et al., 2013), and Calcineurin (Reese and Taglialatela, 2011), are also implicated in linking Aβ and neuronal phenotypes. Signaling that occurs at the mitochondria has also been involved in neuronal dysfunction caused by Aβ. For instance, mPTP dysregulation and abnormal morphology and dynamics of mitochondria were found to be caused by Aβ (Du et al., 2008; Hansson Petersen et al., 2008; Mattson et al., 2008; Wang et al., 2009).

Previously, CentA1 has been shown to be upregulated in the brains of subjects with AD and to accumulate in neuritic plaques (Reiser and Bernstein, 2002, 2004). CentA1 is a brain-specific ADP ribosylation factor 6(Arf6) GTPase activating protein localized to axons, dendrites, dendritic spines and postsynaptic density (Aggensteiner and Reiser, 2003; Moore et al., 2007). CentA1 interacts with the mitochondrial permeability transition pore complex (mPTP) and regulates its function (Galvita et al., 2009). CentA1 interacts with Ras and activates the Ras-Elk-1 pathway, increasing Elk-1-dependent transcription (Hayashi et al., 2006), that can be induced by synaptic activity and neurotrophins (Kalita et al., 2006; Sgambato et al., 1998; Vanhoutte et al., 1999). Elk-1 is also present in extranuclear compartments including dendrites and axons (Sgambato et al., 1998). Extranuclear Elk-1 associates with mPTP in apoptotic neurons (Barrett et al., 2006) and has been implicated in neurodegenerative diseases including AD (Sharma et al., 2010).

There remains a need for therapeutics and methods to prevent memory loss in normal subjects and to treat or ameliorate the symptoms of neurodegenerative diseases in subjects with such a neurodegenerative disease.

### SUMMARY

The invention relates generally to biomedical sciences and more specifically to the field of memory and cognitive improvement in a normal subject and in a subject with brain disorders associated with memory loss and/or cognitive impairments, such as Alzheimer's disease (AD). In particular, the invention relates to CentA1 and proteins involved in neuronal signaling that either modulate CentA1 activity or are modulated by CentA1 as a new therapeutic target to improve memory and cognitive performance in a normal subject, and prevent, treat or slow the progression of memory loss and cognitive impairment associated with dementia, including AD.

In one aspect, the invention relates to CentA1, CentA1 modulating proteins and molecules downstream of CentA1 as targets to improve memory performance.

In another aspect, the invention relates to CentA1, CentA1 interacting proteins and CentA1 downstream effectors as therapeutic targets to prevent and/or treat memory impairment and cognitive impairment in a subject with a brain disorder such as AD and other forms of dementia.

In an embodiment, the invention relates to CentA1, CentA1 interacting proteins and CentA1 downstream effectors as therapeutic targets to prevent and/or treat neurodegeneration associated with AD, Huntington's and Parkinson's diseases.

In one aspect, the invention relates to a screening method for identification of AD related molecules using CentA1 KO mice.

In one aspect, the invention relates to a method of improving memory and cognitive performance in a subject comprising modulating the expression of CentA1, CentA1 effectors, CentA1 interacting proteins and/or downstream signaling molecules including Ras, Arf6, Hsp10, and Elk-1. In one embodiment, the method comprises inhibiting the expression of CentA1, CentA1 effectors, CentA1 interacting proteins, or a combination of two or more thereof.

In another aspect, the invention relates to a method of preventing or treating memory loss in a subject comprising modulating the expression of CentA1, CentA1 effectors, CentA1 interacting proteins and/or downstream signaling molecules including Ras, Arf6, Hsp10, and Elk-1. In one embodiment, the method comprises inhibiting the expression of CentA1, CentA1 effectors, CentA1 interacting proteins, or a combination of two or more thereof.

In yet another aspect, the invention relates to a method of protecting neurons comprising modulating the expression of CentA1, CentA1 effectors, CentA1 interacting proteins and/or downstream signaling molecules including Ras, Arf6, Hsp10, and Elk-1. In one embodiment, the method comprises inhibiting the expression of CentA1, CentA1 effectors, CentA1 interacting proteins, or a combination of two or more thereof.

The methods of the claimed invention may be used in subject that does not have and is not suspected of having a neurodegenerative disease. The methods may also be used in a subject that has, is suspected of having, or is at risk of developing a neurodegenerative disease such as Alzheimer's disease, Huntington's disease, Parkinson's disease, amyotrophic lateral sclerosis, or dementia.

In some embodiments, the method comprises administration of a pharmacological compound to a subject that modulates the expression of CentA1, CentA1 effectors, CentA1 interacting proteins and/or downstream signaling molecules including Ras, Arf6, Hsp10, and Elk-1.

These and other aspects and embodiments are further understood with reference to the following detailed description.

### BRIEF DESCRIPTION OF THE DRAWINGS

FIGS. 1A-1C depict normal brain development in CentA1 KO mice. FIG. 1A depicts images of gels and shows that CentA1−/− hippocampal samples lack an immunoreactive band at the molecular weight of CentA1. FIG. 1B depicts the gross anatomy of CentA1 KO mice at 6 months of age. Coronal sections of the brains were stained with anti-NeuN antibody. FIG. 1C depicts the level of CentA1 in the hippocampal CA1 neurons and cortical neurons in mice at 6 months of age that was evaluated by IHC staining using anti-CentA1 antibody.

FIGS. 2A-2E are graphs comparing long-term memory in mice. FIG. 2A is a line graph comparing the memory acquisition curve over 8 days in Morris water maze test in wild type (WT), homozygous CentA1 (KO) and heterozygous CentA1 (Het) mice. CentA1 KO and wild type mice show a similar memory acquisition curve (age 4-7 months). FIGS. 2B-2C are bar graphs comparing wild type (WT), homozygous CentA1 (KO), and heterozygous CentA1 (Het) mice on day 9 in Morris water maze test. Significant improvement in homozygous CentA1 KO mice were observed in both latency to find platform (FIG. 2B) and number of platform crossings (FIG. 2C), suggesting improved long-term memory. FIGS. 2D-2E are bar graphs comparing wild type (WT), homozygous CentA1 (KO) and heterozygous CentA1 (Het) mice in an open field test to evaluate overall activity and anxiety-like behavior. No significant difference was found among WT, KO and Het in the total distance moved (FIG. 2D) and average velocity (FIG. 2E).

FIGS. 3A-3E demonstrate normal synaptic plasticity and increased dendritic spine density in the hippocampus of CentA1 KO mice. FIG. 3A is a representative image of dendritic spines of Golgi stained CA1 pyramidal neurons from the hippocampus of 4-7 months old wild type and CentA1 KO mice. FIGS. 3B-3D are bar graphs comparing the dendritic spine density of wild type and CentA1 KO mice as evaluated with Golgi staining in CA1 pyramidal neurons in the hippocampus of wild type and CentA1 KO mice at ages 4-7 months (FIG. 3B); 8-10 months (FIG. 3C); and 11-14 months (FIG. 3D). FIG. 3E is graph comparing LTP in wild type and CentA1 KO mice as measured in acute hippocampal slices prepared from mice at 6 months of age.

FIG. 4 is a bar graph comparing apoptotic cell death as evaluated in dissociated hippocampal neuron cultures transfected with scrambled shRNA (shSC) or shRNA against CentA1 and treated with vehicle or Aβ. CentA1 knock down significantly reduced Aβ-induced cell death suggesting neuroprotection against Aβ in neurons lacking CentA1.

FIGS. 5A-5C depict normal brain development in AD model mice (hAPPJ20) lacking CentA1 (APP×KO). FIG. 5A depicts images of gels and shows that APP×KO hippocampal samples lack an immunoreactive band at the molecular weight of CentA1 and express human APP. FIG. 5B depicts the gross anatomy of APP×CentA1 KO mice at 6 months of age. Coronal sections of the brains were stained with anti-NeuN antibody. FIG. 5C depicts deposition of amyloid plaques in the hippocampus of wild type (WT), AD model mice (APP) and AD model mice lacking CentA1 (APP×KO) at 12-14 months of age, evaluated by immunohistochemistry using the 6E10 antibody. FIG. 5D is a bar graph depicting Aβ burden analysis performed at age 12-14 months in the hippocampus of AD model mice and AD model mice lacking CentA1.

FIGS. 6A-6D demonstrate involvement of Ras in Aβ signaling. FIG. 6A shows that Ras activity evaluated by pull-down assay and western blotting is reduced in the hippocampus of CentA1 KO mice compared with wild type litter mates (WT) at 6 months of age. FIG. 6B depicts pull-down assays showing that increased Ras activity in the hippocampus of 6 months old APP mice can be reduced by FTI-276 treatment. FIG. 6C shows that FTI-276 reduces the activity of signaling pathways downstream of Ras as evaluated by western blotting for active/phosphorylated ERK1/2. FIG. 6D is a bar graph comparing dendritic spine density in cultured hippocampal slices treated with either vehicle or Aβ in the presence or absence of FTI-276. Normal density of dendritic spines in CA1 neurons exposed to Aβ is restored by FT-276 treatment.

FIGS. 7A-7F depicts using AD model mice lacking CentA1 to identify new mediators of AD. FIG. 7A depicts 2D-DIGE protein expression profiling of mitochondrial fractions from WT, APP and APP×CentA1 KO mice. Spots denote proteins in the samples. Spot Nr.43 showed a 2 fold increase in APP mice mitochondria compared with WT and was rescued by lack of CentA1. Subsequent mass spectrometry analysis identified with high confidence Spot Nr.43 as Hsp10, a potential new mediator of AD. FIG. 7B shows the level of Hsp10 and its co-chaperone-Hsp60 in the hippocampus of APP mice that was evaluated by western blotting. While Hsp60 level is not affected, Hsp10 is increased in APP brains. FIG. 7C shows representative photomicrographs of CA1 neurons from cultured hippocampal slices transfected with either wild type Hsp10 or empty cloning vector and imaged live using 2 photon microscopy. FIG. 7D is a bar graph depicting that overexpression of Hsp10 reduces spine density in CA1 neurons. FIG. 7E shows representative photomicrographs of CA1 neurons from cultured hippocampal slices transfected with either scrambled shRNA (shSC) or shRNA against Hsp10 and treated for 1 week with either vehicle (Veh) or Aβ42 then imaged live using 2 photon microscopy. FIG. 7F is a bar graph depicting that spine density is rescued from the effect of Aβ by Hsp10 knock down.

The drawings are not to scale unless otherwise noted. The drawings are for the purpose of illustrating aspects and embodiments of the present technology and are not intended to limit the technology to those aspects illustrated therein. Aspects and embodiments of the present technology can be further understood with reference to the following detailed description.

### DETAILED DESCRIPTION

In one aspect, the invention relates to the development and the use of therapeutics for modulating CentA1, CentA1 effectors and CentA1 interacting proteins to improve memory and cognitive performance and delay their decline.

Without limiting the invention to a particular theory, it is believed that CentA1 is a negative regulator of learning and memory and cognitive function of the brain, and decreased CentA1 can improve memory performance and cognitive function in a normal subject and a subject with neurological disorders.

It is believed that Aβ upregulates CentA1 and activates the Ras-Elk-1 pathway at mitochondria, which impairs mitochondrial activity. Analysis of data from the examples described below shows that downregulation of CentA1-Ras-Elk-1 signaling restored normal mitochondrial activity, synaptic function and spine density in Aβ-treated neurons in brain slices.

CentA1 knockout mice were created at Duke Neurobiology Transgenic Core Facility to test this hypothesis. CentA1 KO mice develop with normal weight and there are no detectable gross anatomy changes in the brain structure of the KO mice compared with their wild type litter mates, suggesting that CentA1 is not required for normal development of the brain. Notably, preliminary data suggest that CentA1 KO mice have improved performance in Morris water maze test, a hippocampus-dependent memory test. Consistent with this result, number of spines in the hippocampus was increased in CentA1 KO mice, compared with their wild type litter mates.

These data suggest that inhibition of CentA1 will improve memory performance and cognitive function of the brain. Also, because CentA1 is upregulated in AD brain, the inhibition of CentA1 may improve learning and memory and cognitive functions in a subject with AD or other brain diseases associated with memory loss and cognitive impairment. In addition, in a cellular model of AD, deletion of CentA1 protected neurons from Aβ-induced apoptosis. Thus, inhibition of CentA1 is a potential therapeutic approach in neurodegenerative disorders including AD, Huntington's and Parkinson's diseases.

Because CentA1 KO can reverse cellular phenotypes caused by Aβ, it is possible to use CentA1 KO mice to identify new molecular mediators of AD by performing comparative proteomics studies between wild type, AD model mice and AD model mice crossed with CentA1 KO. Using proteomics screening of the mice with the above background, one can identify new therapeutical targets that are modulated in AD and reversed to normal level in AD mice lacking CentA1. By using this approach Hsp10 was identified as a protein upregulated in the mitochondria of AD model mice and reversed to normal level in APP mice lacking CentA1.

One aspect of the invention relates to a method to improve memory and cognitive performance in normal subjects by means of modulating the activity or expression level of CentA1, CentA1 effectors and/or CentA1 interacting protein.

Memory and cognitive performances can be tested using well established tests, such as evaluation of motor-spatial skills or memory recall testing. The most well established such tests for rodents are Morris Water Maze, Radial Maze, T Maze, and Fear Conditioning. For primates the most well established tests are Eye Blink, Delayed Recall, Cued Recall, and Face Recognition. For humans, there are many established tests for cognitive function and declarative and non-declarative memory using various mazes, pattern recognition tests, condition tasks, etc. Minimental and ADAS-Cog tests are used for cognitive assessment of subjects with AD.

Another aspect of the invention is a method to prevent/reduce memory loss that is associated with normal aging and is exacerbated in dementia, including AD, by means of modulating the activity or expression level of CentA1, CentA1 effectors and/or CentA1 interacting protein.

In one embodiment, the method is a method to prevent/reduce the severity of diseases associated with neurodegeneration, including AD, Huntington's and Parkinson's diseases, by means of modulating the activity or expression level of CentA1, CentA1 effectors and/or CentA1 interacting protein.

In another embodiment, the method is a method to diagnose/prognose the progression of memory loss by evaluating the level of CentA1, CentA1 effectors and CentA1 interacting protein. In one embodiment, the method is a method to diagnose/prognose the progression of AD by evaluating the level of CentA1, CentA1 effectors and CentA1 interacting protein.

The terms “modulating,” “modulation,” “modulated” means upregulation or downregulation of the expression level, or an increase or decrease in the activity of a protein. Modulation also includes regulation of a gene, of the mRNA and any other step in the synthesis or degradation of the protein of interest.

The terms “signaling molecules” and “signaling proteins” are interchangeably used in this application. They are responsible for transmission of information from external stimuli arriving at the cell surface or generated within the cell.

CentA1 is an ADP ribosylation factor 6 (Arf6) GTPase activating protein highly enriched in the brain. In neurons, CentA1 is localized to axons, dendrites, dendritic spines and postsynaptic density (Aggensteiner and Reiser, 2003; Moore et al., 2007). CentA1 interacts with the mitochondrial permeability transition pore complex (mPTP) and regulates its function (Galvita et al., 2009). In a PI3K-dependent manner, CentA1 interacts with Ras and activates the Ras-Elk-1 pathway, increasing Elk-1-dependent transcription (Hayashi et al., 2006).

The term “CentA1 interacting protein” in the context of this disclosure means any protein that directly or indirectly interacts with CentA1 and/or it is either upstream or downstream of the CentA1 signaling pathway.

The term “CentA1 effector” in this context is any protein activated by the CentA1 signaling pathway.

One aspect of the present invention relates to a pharmaceutical composition comprising as an active agent a modulator of CentA1, CentA1 effectors and CentA1 interacting protein together with pharmacologically active diluents, carriers and/or adjuvants. This composition is particularly suitable for improving memory and cognitive performance and delaying the decline of memory and cognitive functions. Depending on the type of inhibitor used as an active agent, the pharmaceutical composition may be a liquid, a solid, e.g. a powder, tablet etc., an emulsion or a suspension. The composition may be administered by injection, orally, topically, rectally, intranasally or by any other suitable means. The effective amount of the active agent in the composition may be determined by the skilled person without any undue burden depending on the type of compound and the disease to be treated.

One aspect of the invention includes a method of administrating a therapeutically effective amount of a composition that modulates CentA1, CentA1 interacting protein and/or CentA1 effector to improve the memory and/or cognitive performance in a subject. The subject may be normal (e.g., does not show signs of neurodegeneration) or may have or may be suspected of having a neurodegenerative disease such as dementia, ALS, AD, Parkinson's, or Huntington's.

One aspect of the invention includes a method of administrating a therapeutically effective amount of a reagent(s) that modulate CentA1, CentA1 interacting protein and/or CentA1 effector to prevent/treat diseases associated with memory and cognitive impairment including AD, as well as delaying memory and cognitive impairment during aging.

Reagents above can be drugs or viruses that contain shRNA or genes encoding mutant forms of CentA1 or regulators of CentA1.

Administration of reagents can be through IP, IV or injection into a ventricle.

One non-limiting example of drugs that modulate CentA1, CentA1 interacting protein and/or CentA1 effector is a farnesyl transferase inhibitor including FTI-276.

In one embodiment of the invention, a therapeutically effective amount of a farnesyl transferase inhibitor is given to a subject to improve memory performance. The subject may be normal (e.g., not show signs of neurodegeneration) or may have or be suspected of having a neurodegenerative disease.

Neurodegenerative diseases include dementia, ALS, Parkinson's, Alzheimer's and Huntington's.

In another embodiment of the invention, a therapeutically effective amount of a farnesyl transferase inhibitor is given to a subject to prevent or treat a neurodegenerative disease.

One non-limiting example of CentA1 effector is Ras (Rat Sarcoma) proteins. Ras proteins are guanosine-nucleotide-binding proteins (G-proteins) involved in cell signaling associated with survival, proliferation and synaptic plasticity in neurons (Ye and Carew, 2010). Ras proteins are a large family including many similar proteins. Ras activation requires association with the plasma membrane via prenylation and palmitoylation. As a first step, the carboxy-terminal CAAX domain of Ras is modified in the cytosol by a farnesyl transferase resulting in addition of 15-carbon isoprenoid (farnesyl group) at the cysteine residue, followed by Ras insertion into the membrane.

Next the AAX tripeptide of Ras is cleaved from the carboxy-terminus of Ras by a prenyl-protein specific endoprotease and the resulting carboxy-terminus is methylated by a methyltransferase. Ras is predominantly localized at the cell membrane due to electrostatic interactions between the positively charged basic sequence of Ras and the negatively charged inner surface of the plasma membrane.

One aspect of the invention includes the use of a Ras inhibitor or a farnesyl transferase inhibitor, including FTI-276, or a combination thereof for improving memory and cognitive performance in normal subjects and preventing/treating diseases associated with memory and cognitive impairment, including AD and other forms of dementia.

In one embodiment, a therapeutically effective amount of a Ras inhibitor or a farnesyl transferase inhibitor, including FTI-276, or a combination thereof is administered for improving memory and cognitive performance. The subject may be normal (e.g., not show signs of neurodegeneration) or may have or be suspected of having a neurodegenerative disease.

In another embodiment of the invention, a therapeutically effective amount of a Ras inhibitor or a farnesyl transferase inhibitor, including FTI-276, or a combination thereof, is given to a subject for preventing/treating diseases associated with memory and cognitive impairment, including AD and other forms of dementia.

“Ras inhibitor” in this disclosure means a compound that can prevent activation of Ras.

“Farnesyl transferase inhibitor” as used herein, means that the Ras inhibitor prevents the addition of a farnesyl group to Ras and therefore translocation of Ras to the plasma membrane. FTI-276 is one example of a farnesyl transferase inhibitor.

Other non-liming examples of CentA1 effectors are Arf6, Hsp10, Hsp60, ERK and Elk-1.

The invention includes the use of reagents that modulate Arf6, Hsp10, Hsp60, ERK and Elk-1 for improving memory and cognitive performance in normal subjects and preventing/treating diseases associated with memory and cognitive impairment, including AD and other forms of dementia.

In one embodiment, a therapeutically effective amount of a reagents that modulates Arf6, Hsp10, Hsp60, ERK or Elk-1, or a combination thereof, is administered for improving memory and cognitive performance. The subject may be normal (e.g., not showing signs of neurodegeneration) or may have or be suspected of having a neurodegenerative disease.

In another embodiment of the invention, a therapeutically effective amount of a reagents that modulates Arf6, Hsp10, Hsp60, ERK or Elk-1, or a combination thereof, is administered for preventing/treating diseases associated with memory and cognitive impairment, including AD and other forms of dementia.

One aspect of the invention includes the use of CentA1, CentA1 interacting protein and/or a CentA1 effector to diagnose or prognose dementia including Alzheimer's disease. The term “use” means any use of these proteins or their mRNA or the genomic DNA to detect mutations or to analyze expression level of the translated proteins. Methods for mutation analysis, expression analysis and detection and quantification of protein are routine laboratory techniques.

In another aspect, also disclosed is the use of CentA1 knockout mice to identify proteins that are modulated by AD and CentA1. Inhibitors targeting the identified proteins potentially can be a new therapeutic target for AD. Examples of assays to identify new drug targets are described in the Example section of this disclosure. There is no specific order in performing the proposed experiments.

To further understanding of this disclosure, we are presenting the following examples:

### EXAMPLES

### Example 1

### CentA1 KO Mice have No Developmental Deficit when Compared with Wild Type Litter Mates

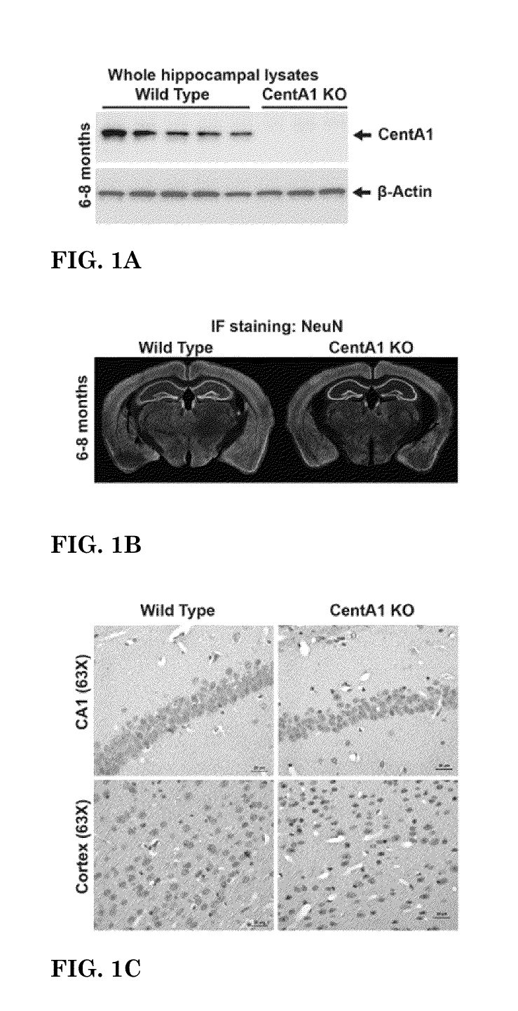

CentA1 KO mice develop with normal weight (34.4±1.8 g for WT [N=5], 36.9±2.6 g for KO [N=4] at age 4-7 months). There are no detectable gross anatomy changes in the brain structure of the KO mice compared with their wild type litter mates (FIG. 1B).

### Example 2

### Improved Memory Performance in Mice Lacking CentA1 Gene

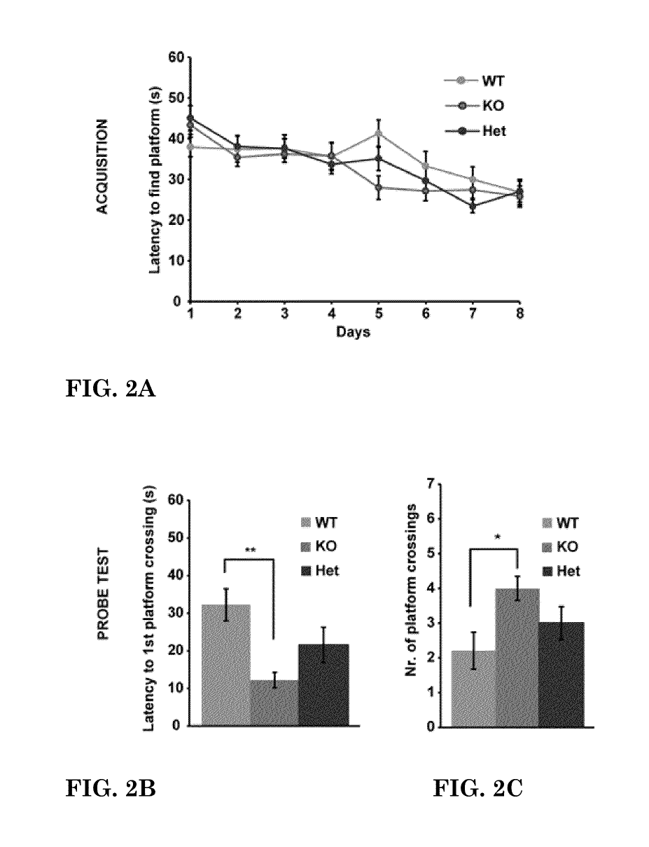

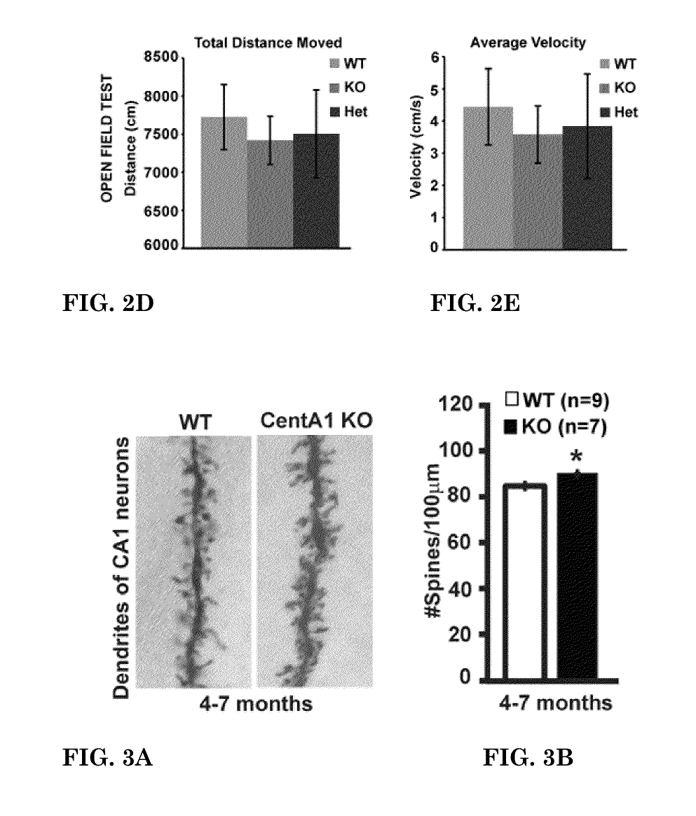

Behavioral assays on CentA1 KO mice were performed at the Scripps Florida Institute Behavioral Core Facility, which is located next to Max Planck Florida Institute for Neuroscience. A battery of behavioral tests were performed including: elevated plus maze (EPM) to test anxiety-like behavior, Rota-Rod to test motor coordination, open field to test activity and anxiety-like behavior, Y-maze spontaneous alternation to test working memory, Morris water maze to test spatial memory acquisition, and classical fear conditioning to test fear memory. Lack of CentA1 does not cause detectable changes in most of these tests. However, long-term memory recall evaluated by Morris water maze test was significantly improved (FIGS. 2B-C). Increased memory performance may be caused by increased density of synapses. Golgi staining on mice at different ages (4-7 months and 8-12 months) was performed and the spine density of pyramidal neurons in hippocampus and neocortex was measured. Data suggest that there is a significant age-dependent increase in dendritic spine density in hippocampal CA1 pyramidal neurons of CentA1 KO mice compared to wild type litter mates (FIGS. 3A-D). Increased memory performance may be associated with increased synaptic plasticity. To test this hypothesis, LTP in acute hippocampal slices prepared from mice at age 4-7 months was measured. Data show that there is no difference in LTP between genotypes (FIG. 3E).

### Example 3

### Lack of CentA1 Provides Neuroprotection Against Aβ-Mediated Neurotoxicity

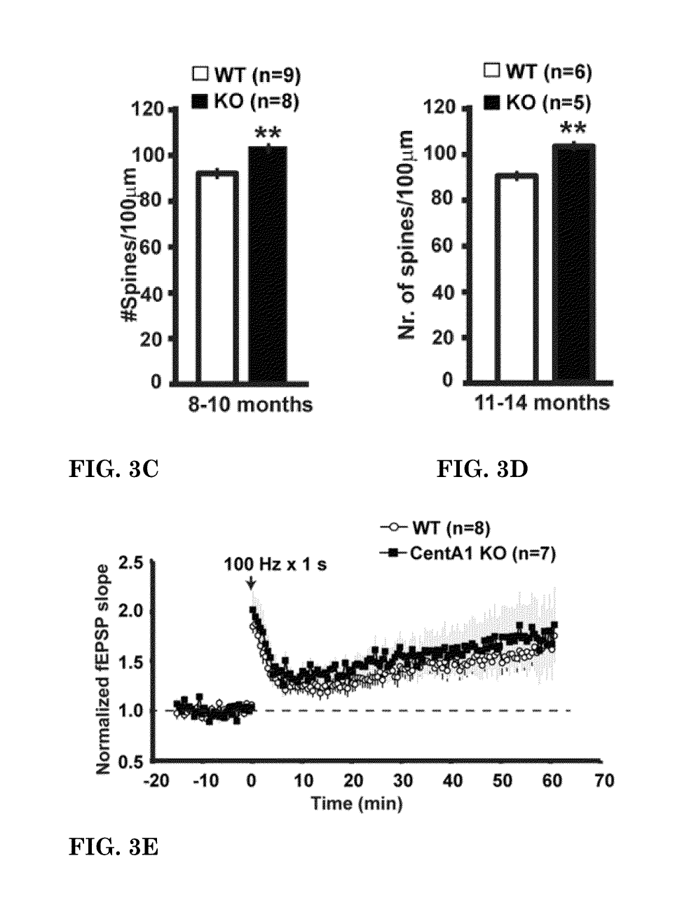

Apoptotic cell death was observed in human brain with AD and in the brain of transgenic mouse models of AD (Absalon et al., 2013; Chong et al., 2006; Eckert et al., 2011; Ghavami et al., 2014; Gotz et al., 2011; Ivins et al., 1998). To evaluate whether reduced CentA1 expression protects against Aβ-mediated neurotoxicity, shRNA was used against CentA1. A significant reduction in apoptotic cell death by Aβ in dissociated hippocampal neuron cultures was observed (FIG. 4).

### Example 4

### Generation of APP×CentA1 KO Mice

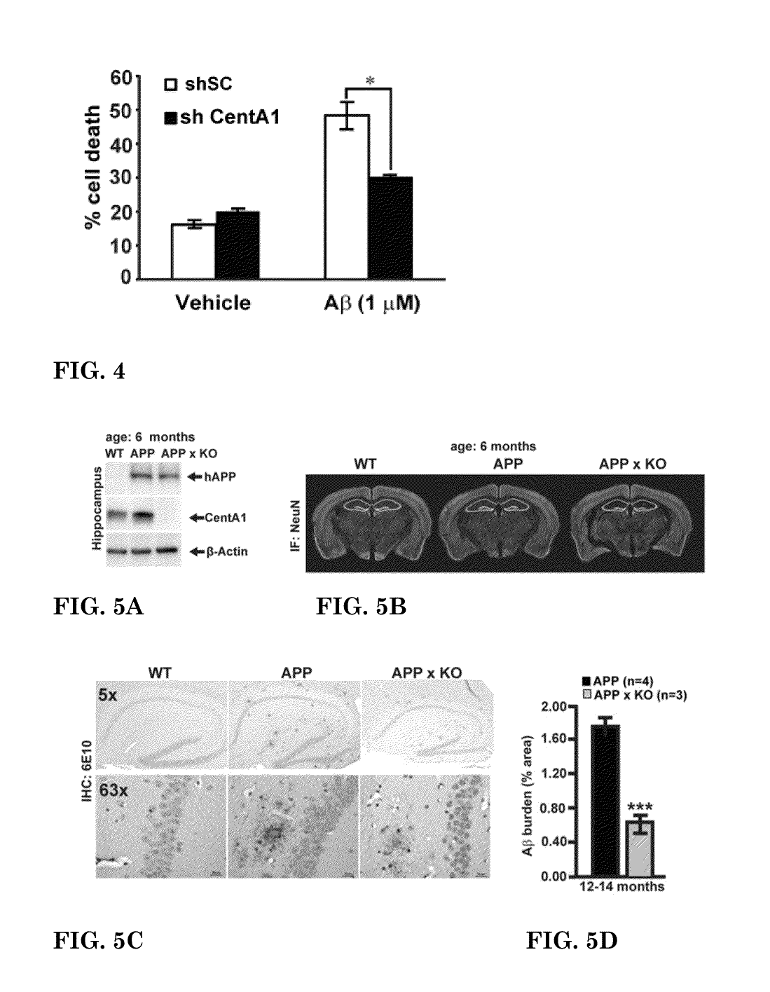

To directly address the role of CentA1 in pathogenesis of AD, CentA1 KO mice were crossed with hAPPJ20 model mice (APP×KO). APP×KO mice develop normally and there are no detectable gross anatomy changes in the brain structure of the APP×KO mice compared with APP and wild type litter mate mice (FIG. 5B). Aβ burden analysis performed at age 12-14 months, shows significant reduction in amyloid burden in the hippocampus of AD model mice lacking CentA1 (FIG. 5D).

### Example 5

### Inhibition of Ras Signaling Protects Against Aβ-Mediated Neurotoxicity

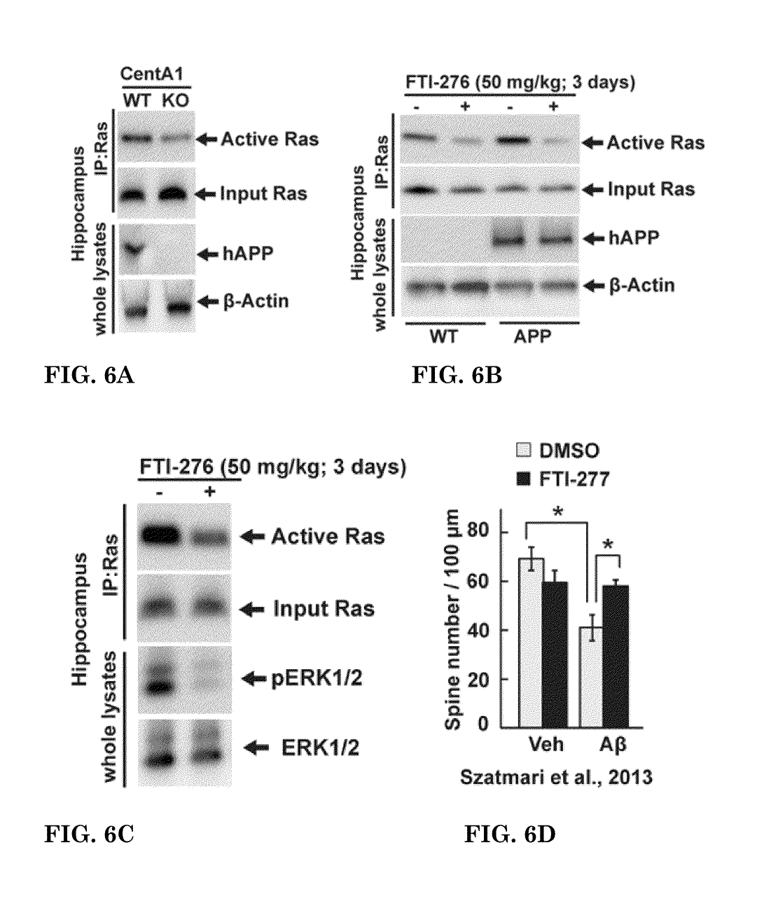

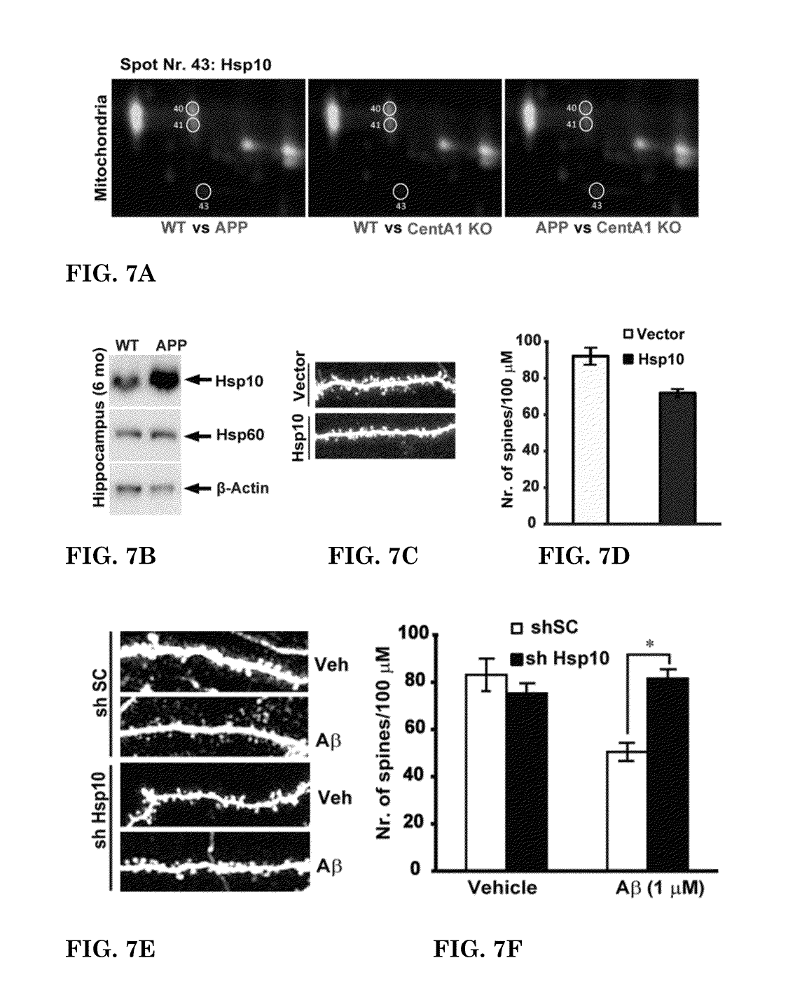

Recent studies indicate a requirement of Ras-ERK signaling in neurodegeneration. For instance downregulation of the Ras-ERK-MSK pathway was found to be protective against neurodegeneration in Drosophila and mice (Park et al., 2013). Transgenic mouse models of AD exhibit increased Ras activity; while in APP KO mice Ras activity is significantly reduced (Mora et al., 2013a; Mora et al., 2013b; Song et al., 2014). Use of a farnesyltransferase inhibitor, FTI-277, prevented some Aβ-induced phenotypes in organotypic slices including spine loss (FIG. 7F) and decreased mEPSC frequency (Szatmari et at, 2013). Active Ras pull down assay shows reduced Ras activity in CentA1 KO mice (FIG. 6A). Moreover daily IP injections of FTI-276 for 3 consecutive days prevented Ras hyperactivation in AD model mice (FIG. 6B) and reduced phosphorylation/activation of ERK1/2 that is downstream of Ras signaling (FIG. 6C). Thus, farnesyltransferase inhibitors may be used in potential therapeutic strategies to prevent/delay AD progression.

### Example 6

### Proteomic Screening of Transgenic Mice Identifies Potential Drug Targets for AD Therapy

To identify new signaling molecules activated by CentA1 signaling in AD, protein profiling using two-dimensional difference in-gel electrophoresis (2D-DIGE) followed by mass spectrometry (MALDI-TOF) of wild type, AD model mice and AD model mice lacking CentA1 was performed. Using this approach, upregulation of Hsp10 in the mitochondrial fractions of AD model mice was found. This was reversed to normal level in APP mice lacking CentA1. Heat shock protein 10 (Hsp10) was first identified as a mitochondria specific chaperone that forms with Hsp60 a molecular complex required for folding of mitochondrial proteins (Kahle et al., 2013; Park et al., 2013). Although few studies indicate involvement of Hsp10 in normal aging (Ebner et at, 2013), the role of this chaperon in age-related disorders has not been studied. The hypothesis that Hsp10 is involved in the pathogenesis of Alzheimer's disease was tested. Overexpression of Hsp10 in organotypic hippocampal slices reduced dendritic spine density, while shRNA mediated Hsp10 knockdown protected CA1 hippocampal neurons from Aβ-mediated spine loss (FIGS. 7D and 7F).

### Materials and Methods to the Examples

### Animals

As a model for AD, male hAPPJ20 transgenic mice were used (Mucke et al., 2000). Non-transgenic male littermates were used for controls.

To generate CentA1 KO mice, exon 3 (functional domain) of the Centa1/Adap1 locus was replaced with sequences for LacZ, followed by a translational STOP, and a neo selection cassette flanked by frt sites. The neo selection cassette was removed by crossing with Flp line. Expression and translation of this modified locus results in a fusion protein of the beginning of CentA1 (exon 1 and 2) and LacZ and lack of the functional CentA1 protein.

Mouse studies were approved by the institutional animal care and use committee of Max Planck Florida Institute in accordance with the National Institutes of Health guidelines for animal care.

### Preparations

Dissociated hippocampal cultures were prepared from newborn Sprague Dawley rats at postnatal day 0-1. Neurons were cultured in basal medium Eagle (BME) supplemented with 10% heat-inactivated fetal bovine serum (HyClone), 35 mm glucose, 1 mm 1-glutamine, 100 U/ml penicillin, and 0.1 mg/ml streptomycin. Cytosine arabinoside (2.5 μm) was added to the cultures on day(s) in vitro (DIV) 2 to inhibit the proliferation of non-neuronal cells. Neurons were transfected by electroporation using the Amaxa Nucleofector System (Lonza Bioscience Cell Discovery) or by lipofection.

Organotypic hippocampal slice cultures (300 μm thick) were prepared from mice at postnatal day 6 or 7. After 1 week in culture, slices were transfected using biolistic gene transfer. Aβ stock solution (200 μM) was prepared in 1% NH4OH as a vehicle and added directly to the culture medium at a final concentration of 1 μM. FTI-277 was dissolved in DMSO and added directly to the culture medium at a final concentration of 10 μM.

### Behavioral Studies

### Morris Water Maze:

Spatial learning was examined using the Morris water maze (Taylor et al., 2008). Performance was assessed by visual analyses of the tracking profiles for each animal and the swim distance, swim time, and swim velocity to find the hidden platform were determined by Noldus Ethovision (Noldus Information Technology Inc., Leesburg, Va.). Testing was conducted in pool divided into quadrants labeled northeast (NE), northwest (NW), southeast (SE), and southwest (SW) relative to a camera positioned 140 cm above the center of the pool. A white metal platform was located approximately 1 cm below the surface of the water and 20 cm from the edge of the pool in the NE quadrant. Testing was conducted under two types of conditions: acquisition in which the platform was hidden below the surface of the water in the NE quadrant and probe trials where the platform was removed from the maze. All test trials were 1 min in duration. Acquisition testing consisted of 32 trials given across 8 days with 4 trials administered per day. Trials were run in pairs, with each pair separated by 60 min. Probe tests were given at the end of day 9.

### Spontaneous Activity in the Open Field:

Open field behavioral testing was performed as previously described (Pogorelov et al., 2005). Briefly, mice were evaluated in 17×17 in. square acrylic open field chambers. Before testing, uniformity of light across the arena was confirmed using a light intensity meter. The chambers were cleaned with 1% Micro-90 before and between trials. Background white noise (approximately 72 dB) was used during trials. Mice were placed into the center of the chamber and activity was recorded for 30 min. Data were analyzed in 10 min blocks.

### Evaluation of Dendritic Spine Density:

Golgi staining of mouse brain was performed using FD Rapid GolgiStain kit, following the instructions of the manufacturer. Dendritic spine density on apical dendrites of hippocampal CA1 neurons was analyzed on 100 μm thick brain sections using Image J software.

### Fluorescent Immunostaining:

Mice were anesthetized with Ketamine/Xylazine cocktail and perfused transcardially with PBS, followed by 4% paraformaldehyde. The brain was postfixed in 4% paraformaldehyde for 24 hours, and then transferred into 30% sucrose in PBS for 48 hours. Coronal sections from the brains were cut at 50 μm on a vibratome (Leica). Sections were blocked (0.5% goat serum, 0.3% TX-100 in PB) for 30 minutes, and then incubate with rabbit anti-NeuN antibody (Millipore; 1:1000) in blocking solution for 24 hours at 4 C. Next, brain sections were rinsed with PB (3×15 minutes) and incubated with Alexa 488-coupled goat anti-rabbit IgG in PBS (Life Technologies; 1:500) for 2 hours at room temperature. Sections were rinsed with PB (3×15 minutes), then mounted on slides and coverslipped with fluorescent mounting medium (Vector Laboratories).

### Immunohistochemistry (IHC):

For IHC studies, mice were deeply anesthetized with Ketamine/Xylazine cocktail until lack of response to toe pinch was recorded. After transcardial perfusion with 50 mL PBS and 50 mL 4% paraformaldehyde (PFA), the brain was harvested and post-fixed in 4% PFA for at least 24 hours. Paraffin embedded sagittal brain sections (5 μm thickness) were mounted on Superfrost Plus slides (Fisherbrand). Sections were deparaffinized, and then either pretreated with 70% Formic Acid (6E10, Covance) or heat retrieved in a Biocare Decloaker (anti-Centaurin α-1, Abcam). For blocking we used Rodent Block M (Biocare Medical, Concord, Calif.) for 30 minutes at room temperature. Samples were incubated overnight at 4° C. with primary antibody diluted in Van Gogh Diluent (Biocare Medical, Concord, Calif.): 1:4000 (6E10, Covance) and 1:1000 (anti-Centaurin α-1, ab27476, Abcam). Sections were rinsed with TBS-T and incubated with secondary HRP-Polymers (Mouse-on-Mouse or Goat-on-Rodent; Biocare Medical, Concord, Calif.) for 30 minutes at room temperature. After several TBS-T rinses, samples were reacted with Betazoid DAB (Biocare Medical, Concord, Calif.), followed by TBS washes, stained with Hematoxylin, dehydrated and coverslipped with Leica Micromount mounting medium.

### Aβ Plaque Burden Analysis:

Brain sections of 5 μm thickness, cut at 30 μm apart (3 sections/mouse; N=3-4/genotype) were IHC stained with the 6E10 antibody (human Aβ1-16 specific). Aβ burden (% area) in the hippocampus of APP and APP×KO mice was quantified using the positive pixel count program (Aperio).

### Electrophysiology:

For LTP induction, organotypic hippocampal slices isolated from 6-month old male mice were used. Briefly, mice were sedated with isoflurane and perfused intracardially with a chilled choline chloride solution. Brain was removed and placed in the same choline chloride solution (124 mM Choline Chloride, 2.5 mM KCl, 26 mM NaHCO3, 3.3 mM MgCl2, 1.2 mM NaH2PO4, 10 mM Glucose and 0.5 mM CaCl2, pH 7.4 equilibrated with 95% O2/5% CO2). Coronal slices (400 μm) were prepared and maintained in a submerged chamber at 32° C. for 1 h and then transferred to room temperature in oxygenated ACSF. Extracellular field potentials were recorded using 5-8 mΩ glass micropipettes filled with ACSF. A concentric bipolar stimulation electrode and glass micropipette were placed into the stratum Radiatum of the CA1 area at approximately 300 μm apart and at the same distance from the pyramidal cell layer and same depth. Signals were amplified at gain 100, filtered at 2 kHz and digitized at 20 kHz. An input/output curve was recorded to determine the stimulation amplitude, defined by the stimulus intensity that elicits a fEPSP that is 50% of the supramaximal response. Stimulation was applied every 20 s and the baseline was recorded for 20 min or until stabilization of the fEPSP amplitude. LTP was induced by high frequency stimulation protocol consisting of 100 Hz. Post-stimulus fEPSPs were then recorded for 1 hour. All data was analyzed with an in-house program written with Matlab.

### Isolation of Mitochondrial Proteins:

Mitochondria from mouse organotypic hippocampal slices were isolated using mitochondrial isolation kit for tissue (Thermo Scientific). Proteomic evaluation of mitochondria was performed at Applied Biomics (Hayward, Calif.). First, mitochondrial extracts were subjected to two-dimensional difference in-gel electrophoresis (2D-DIGE). Protein spots with a 2-fold change between genotypes were identified by MALDI-TOF.

### Ras Activity Assay:

The level of active Ras in hippocampal lysates from CentA1 KO mice and their wild type litter mates was measured with an active Ras pull-down and detection kit and following the instructions from manufacturer (Thermo Scientific). Western blotting was conducted with mouse anti-Ras antibody.

### Western Blot Analysis:

Protein expression and/or phosphorylation was evaluated by standard western blotting protocol using the following antibodies: goat anti-centaurin-α1 (Abcam; 1:500); mouse anti-β-actin (Sigma, 1:2000); mouse 6E10 (Covance, 1:1000); rabbit anti-phosphoERK1/2 (Cell Signaling Technologies, 1:1000); mouse anti-ERK1/2 (Cell Signaling Technologies, 1:1000); mouse anti-Ras (Pierce, 1:1000); rabbit anti-Hsp10 (Cell Signaling Technologies, 1:1000); rabbit anti-Hsp60 (Cell Signaling Technologies, 1:1000); and HRP-labeled anti-mouse, anti-goat, or anti-rabbit IgG antibodies (BioRad; 1:2000).

### Quantitation of Neuronal Death Based on Change in Nuclear Morphology:

Cells were stained with Hoechst 33258 and observed using fluorescent microscopy. Cells with condensed or fragmented nuclei were scored as apoptotic. For transfected neurons, cell death was scored in β-gal-positive cells. At least 150 cells were analyzed for each condition in each experiment.

Embodiments of the technology have been described above and modifications and alterations may occur to others upon the reading and understanding of this specification. The claims as follows are intended to include all modifications and alterations insofar as they come within the scope of the claims or the equivalent thereof.

What is claimed is:

1. A method of improving memory and cognitive performance in a subject comprising modulating the expression of CentA1, CentA1 effectors, CentA1 interacting proteins and/or downstream signaling molecules including Ras, Arf6, Hsp10, and Elk-1.

2. The method of claim 1, wherein improving memory and cognitive performance comprises inhibiting the expression of CentA1, CentA1 effectors, CentA1 interacting proteins, or a combination of two or more thereof.

3. The method of claim 1, wherein the subject does not have and is not suspected of having a neurodegenerative disease associated with memory loss or cognitive impairment.

4. The method of claim 1, wherein the subject has or is suspected of having a neurodegenerative disease associated with memory loss or cognitive impairment.

5. The method of claim 4, wherein the neurodegenerative disease is selected from Alzheimer's disease, Huntington's disease, Parkinson's disease, amyotrophic lateral sclerosis, or dementia.

6. The method of claim 2, wherein inhibiting the expression comprises administering a pharmacological compound comprising at least one farnesyl transferase inhibitor.

7. The method of claim 6, wherein the pharmacological compound comprises FTI-276.

8. A method of preventing or treating memory loss in a subject comprising modulating the expression of CentA1, CentA1 effectors, CentA1 interacting proteins and/or downstream signaling molecules including Ras, Arf6, Hsp10, and Elk-1.

9. The method of claim 8, wherein preventing or treating memory loss comprises inhibiting the expression of CentA1, CentA1 effectors, CentA1 interacting proteins, or a combination of two or more thereof.

10. The method of claim 8, wherein the subject does not have and is not suspected of having a neurodegenerative disease associated with memory loss or cognitive impairment.

11. The method of claim 8, wherein the subject has or is suspected of having a neurodegenerative disease associated with memory loss or cognitive impairment.

12. The method of claim 11, wherein the neurodegenerative disease is selected from Alzheimer's disease, Huntington's disease, Parkinson's disease, amyotrophic lateral sclerosis, or dementia.

13. The method of claim 9, wherein inhibiting the expression comprises administering a pharmacological compound comprising at least one farnesyl transferase inhibitor.

14. The method of claim 13, wherein the pharmacological compound comprises FTI-276.

15. A method of protecting neurons by modulating expression of CentA1, CentA1 effectors, CentA1 interacting proteins and/or downstream signaling molecules including Ras, Arf6, Hsp10 and Elk-1.

16. The method of claim 15 comprising inhibiting the expression of CentA1, CentA1 effectors, CentA1 interacting proteins, or a combination of two or more thereof.

17. The method of claim 16, wherein inhibiting the expression comprises administering a pharmacological compound comprising FTI-276 to a subject.

18. The method of claim 17, wherein the subject does not have and is not suspected of having a neurodegenerative disease associated with memory loss or cognitive impairment.

19. The method of claim 17, wherein the subject has or is suspected of having a neurodegenerative disease associated with memory loss or cognitive impairment.

20. The method of claim 19, wherein the neurodegenerative disease is selected from Alzheimer's disease, Huntington's disease, Parkinson's disease, amyotrophic lateral sclerosis, or dementia.
